(19) United States
(12) Patent Application Publication
(10) Pub. No.: US 2013/0078185 A1
(43) Pub. Date: Mar. 28, 2013

(54) NITROIMIDAZOLE DERIVATIVES

(71) Applicants: Vijaya Raj Kuniyil Kulangara, Bangalore (IN); Chandan Ramaswamy Atreya, Bangalore (IN)

(72) Inventors: Vijaya Raj Kuniyil Kulangara, Bangalore (IN); Chandan Ramaswamy Atreya, Bangalore (IN)

(73) Assignee: GE HEALTHCARE LIMITED, BUCKINGHAMSHIRE (GB)

(21) Appl. No.: 13/700,187

(22) PCT Filed: May 31, 2011
(86) PCT No.: PCT/EP11/58938

§ 371 Date: Nov. 27, 2012

(30) Foreign Application Priority Data
May 31, 2010 (IN) 1251/DEL/2010
Jul. 21, 2010 (GB) 1012209.1

(51) Int. Cl. A61K 51/04; C07B 59/00; C07F 7/22; C07D 498/04

(52) U.S. Cl. CPC A61K 51/0453; C07D 498/04; C07B 59/002; C07F 7/2212
USPC 424/1.85; 548/218; 548/103

(57) ABSTRACT

The present invention provides novel compounds useful in the treatment and diagnosis of mycobacterial infections. Compounds of the present invention have enhanced biological properties as compared to the related known compounds. The present invention also provides a precursor compound useful in the synthesis of certain compounds of the invention, and a method to obtain these compounds using said precursor compound. Methods of treatment and diagnosis in which the compounds of the invention fmd use are also provided.

### TECHNICAL FIELD OF THE INVENTION

The present invention relates to compounds having activity against mycobacteria. Certain compounds of the invention may be used in the treatment of mycobacterial infections. The invention also provides radiolabelled compounds that are useful for in vivo imaging in the diagnosis of mycobacterial infections. Methods and intermediates useful for the preparation of certain compounds of the invention are also provided. The invention also provides methods for using the compounds of the invention in treatment and diagnosis.

### DESCRIPTION OF RELATED ART

Pulmonary tuberculosis (TB) is an airborne infection caused by Mycobacterium tuberculosis (MTB) that causes high mortality and morbidity, particularly in developing countries (Dye et al JAMA 1999; 282(7): 677-686). A recent factsheet produced by the World Health Organisation reported that the number of new cases of TB continues to increase each year in South-East Asia, the Eastern Mediterranean and Africa (http://wwww.who.int/mediacentre/factsheets/fs104/en/print.html). The antitubercular nitroimidazoles, including two classes of new bicyclic agents with either fused oxazole or oxazine rings, are one of the most exciting recent developments in the field of antituberculosis chemotherapy, and two candidates are already in human clinical trials for the treatment of both drug-susceptible and drug-resistant disease (in this regard the reader is referred to the website http://www.newtbdrugs.org/pipeline.php). Sasaki et al (J Med Chem 2006; 49(26): 7854-7860) have reported a series of novel optically active 6-nitro-2,3-dihydroimidazo oxazoles having various phenoxymethyl groups and a methyl group at the 2-position. A particular compound that is potent and orally active was found that is a promising candidate (OPC-67683) for the treatment of tuberculosis, which is currently in clinical trials:

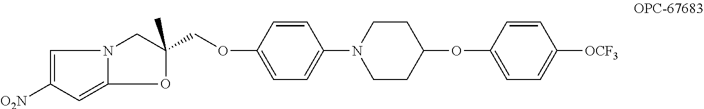 [SMILES: C[C@]1(COc2ccc(N3CCC(Oc4ccc(OC(F)(F)F)cc4)CC3)cc2)Cn2cc([N+](=O)[O-])cc2O1]

The unique structure of the cell wall of mycobacteria, rich in waxy mycolic acid, is the target of action of OPC-67683, which inhibits methoxy-mycolic and keto-mycolic acid synthesis but at significantly lower concentrations.

With the recent emergence of drug-resistant strains of MTB there is still scope for further improved agents to treat an otherwise incurable disease.

Radiolabelled nitroimidazoles are well-known for hypoxia imaging. Examples include ¹F-misonidazole ([¹⁸F]FMISO) and ^{99m}TcO(PnAO)-1-2-nitroimidazole (known as BMS-181321):

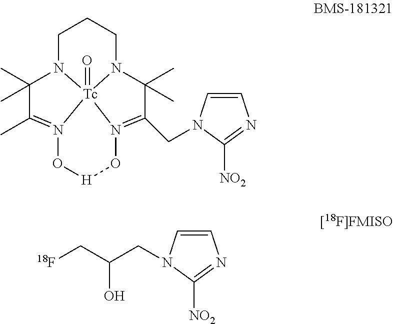

These and other radiolabelled nitroimidazoles have been described as being particularly useful in the detection of myocardial hypoxia (Strauss et at J Nuc Cardiol 1995; 2: 437-445).

Accurate and prompt diagnosis is important in order to control the infection and also to ensure the appropriate therapy for infected patients. Currently, a definitive diagnosis of TB requires culture of MTB from a sample taken from a patient. Patients with clear signs and symptoms of pulmonary disease with a sputum smear-positive result present no problems to diagnose. However, there can be difficulty culturing the slow-growing MTB organism in the laboratory. Furthermore the emergence of HIV has resulted in a decreased likelihood of sputum smear positivity and an increase in non-respiratory disease, such that ease of diagnosis is more difficult in these cases (see reviews by Jeong & Lee Am J Roent 2008; 191: 834-844; Davies & Pai Int J Tuberc Lung Dis 2008; 12(11): 1226-1234; and, Lange & Mori Respirology 2010; 15: 220-240).

In vivo imaging methods are known to be useful in the diagnosis of TB. Chest x-ray is a widely-used in vivo imaging method for screening, diagnosis and treatment monitoring in patients with known or suspected TB. Chest computed tomography (CT) is more sensitive than conventional x-ray and may be applied to identify early parenchymal lesions or mediastinal lymph node enlargements and to determine disease activity in tuberculosis (Lee & Im AJR 1995; 164(6): 1361-1367).

Nuclear imaging methods have also been reported for diagnosis and treatment monitoring of TB. The positron-emission tomography (PET) tracer ¹⁸F-fluorodeoxyglucose ([¹⁸F]FDG) has been proposed as useful in the diagnosis of disease activity and therapy monitoring in patients with TB (Demura et at Eur J Nuc Med Mol Imag 2009; 36: 632-639). Roohi et at (Radiochim Acta 2006; 94: 147-152) describe a ^{99m}Tc-labelled isoniazid derivative, which localised to tubercular lesions in rabbits and enabled the lesions to be visualised 2 hours following administration of the ^{99m}Tc-labelled derivative. However, this ^{99m}Tc-labelled derivative comprises a ^{99m}Tc-chelate at a location believed to be the active pharmacophore, which is not ideal.

There is therefore scope for improved strategies in the treatment and diagnosis of TB.

### SUMMARY OF THE INVENTION

The present invention provides novel compounds useful in the treatment and diagnosis of mycobacterial infections. Compounds of the present invention have enhanced biological properties as compared to the related known compounds. The present invention also provides a precursor compound useful in the synthesis of certain compounds of the invention, and a method to obtain these compounds using said precursor compound. Methods of treatment and diagnosis in which the compounds of the invention find use are also provided.

### DETAILED DESCRIPTION OF THE INVENTION

Compound

In one aspect, the present invention provides a compound of Formula I:

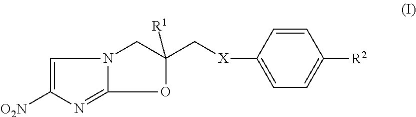

wherein:

R¹ is absent or is C₁₋₄ alkyl;

R² is a halogen isotope; and,

X is —O— or —NH—.

Unless otherwise specified, the term “alkyl” alone or in combination, means a straight-chain or branched-chain alkyl radical containing preferably from 1 to 4 carbon atoms. Examples of such radicals include, methyl, ethyl, and propyl.

The term “halogen isotope” refers to any radioactive or non-radioactive isotope of a halogen (also referred to herein as “radioactive halogen” and “non-radioactive halogen”, respectively). The terms radioactive and non-radioactive take their commonly-known meaning, i.e. “radioactive” refers to giving off, or capable of giving off, radiant energy in the form of particles or rays, as alpha, beta, and gamma rays, by the spontaneous disintegration of atomic nuclei. The term “non-radioactive” means not radioactive. The term “halogen” suitably refers to an atom selected from iodine, fluorine, chlorine and bromine, preferably to iodine and fluorine and most preferably to iodine.

R¹ is preferably C₁₋₄ alkyl, and is most preferably methyl.

X is preferably —O—.

In one preferred embodiment, R² is a gamma-emitting radioactive halogen selected from ¹²³I, ¹³¹I and ⁷⁷Br. For this embodiment said gamma-emitting radioactive halogen is preferably ¹²³I.

In another preferred embodiment, R² is a positron-emitting radioactive halogen selected _{from} ¹⁷F, ¹⁸F, ⁷⁵Br, ⁷⁵Br and ¹²⁴I. For this embodiment, said positron-emitting radioactive halogen is selected from ¹⁸F and ¹²⁴I, and is most preferably ¹²⁴I.

In a further preferred embodiment, R² is a non-radioactive halogen selected from ¹²⁷I, ⁷⁹Br, ⁸¹Br, ¹⁹F. For this embodiment, said non-radioactive halogen is preferably selected from ¹²⁷I and ¹⁹F, and is most preferably ¹²⁷I.

If a chiral centre or another form of an isomeric centre is present in a compound according to the present invention, all forms of such isomer, including enantiomers and diastereoisomers, are encompassed by the present invention. Compounds of the invention containing a chiral centre may be used as racemic mixture or as an enantiomerically-enriched mixture, or the racemic mixture may be separated using well-known techniques and an individual enantiomer maybe used alone. In a preferred embodiment, an individual enantiomer is used alone. Preferably, individual enantiomer of the compound as defined herein is of Formula Ia:

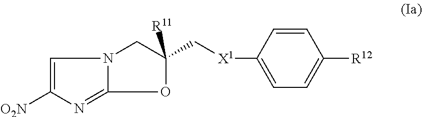

wherein R¹¹, R¹² and X¹ are as suitably and preferably defined herein for R¹, R² and X, respectively.

Precursor Compound

In another aspect, the present invention provides a precursor compound for the preparation of compound of Formula I wherein R² is a radioactive halogen as defined above, wherein said precursor compound is a compound of Formula II:

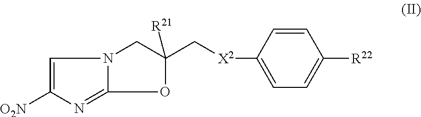

wherein:

R²¹ is as defined above for R¹ of Formula I;

R²² is a non-radioactive iodine or bromine, an organometallic derivative such as a trialkylstannane or a trialkylsilane, an organoboron compound such as a boronate ester or an organotrifluoroborate, or is selected from amino, hydroxy, nitro, bromo, iodo, tri-C₁₋₃-alkylammonium, quaternary ammonium, diazonium, iodonium, tosylate, mesylate and triflate; and,

X² is as defined above for X of Formula I.

A “precursor compound” comprises a non-radioactive derivative of a radiolabelled compound, designed so that chemical reaction with a convenient chemical form of the detectable label occurs site-specifically; can be conducted in the minimum number of steps (ideally a single step); and without the need for significant purification (ideally no further purification), to give the desired radiolabelled compound. In the context of the present invention, the term “radiolabelled compound” refers to the compound of Formula I wherein R² is a radioactive halogen. Such precursor compounds are synthetic and can conveniently be obtained in good chemical purity. In order to facilitate site-specific reaction, the precursor compound of the invention may optionally comprise a suitable protecting group.

By the term “protecting group” is meant a group which inhibits or suppresses undesirable chemical reactions, but which is designed to be sufficiently reactive that it may be cleaved from the functional group in question to obtain the desired product under mild enough conditions that do not modify the rest of the molecule. Protecting groups are well known to those skilled in the art and are described in ‘Protective Groups in Organic Synthesis’, Theorodora W. Greene and Peter G. M. Wuts, (Fourth Edition, John Wiley & Sons, 2007).

An “organometallic derivative” is an organic substituent containing a metal, especially a wherein a metal atom is bonded directly to a carbon atom. In the context of the present invention the term preferably relates to trialkylstannane and trialkylsilane substituents. The term “trialkylstannane” refers to the moiety —Sn-(alkyl)₃, wherein each alkyl is the same and wherein the term alkyl is as defined above, and is preferably a C₁₋₆ alkyl, most preferably methyl or butyl, and most especially preferably butyl. The term “trialkylsilane” refers to the moiety —Si-(alkyl)₃ wherein the (alkyl)₃ portion is as defined for trialkylstannane.

The term “organoboron compound” (also known as organoborane compound) refers to a substituent that is an organic derivative of BH₃. A “boronate ester” is a substituent derived from an alkyl or aryl substituted boric acid containing a carbon-boron bond belonging to the larger class of organoboranes, wherein the terms alky and aryl are as defined herein. An “organotrifluoroborate” is a substituent derivaed from an organoboron compound that contains an anion with the general formula [RBF₃]⁻.

The term “amino” refers to the group —NH₂.

The term “hydroxyl” refers to the group —OH.

The term “nitro” refers to the group —NO₂.

The term “bromo” refers to a bromine substituent.

The term “iodo” refers to an iodine substituent.

The term “quaternary ammonium” refers to the group —NR₃ wherein each R is an alkyl or an aryl, wherein the terms alkyl and aryl are as defined herein. Preferably, each R is an alkyl, most preferably a C₁₋₃ alkyl.

The term “diazonium” refers to the —N⁺≡N group.

The term “iodonium” in the context of the present invention refers to the ion RI⁺ wherein R is any organic residue. R is preferably an aryl wherein the term “aryl” refers to aromatic rings or ring systems having 5 to 12 carbon atoms, preferably 5 to 6 carbon atoms, in the ring system, e.g. phenyl or naphthyl.

The term “tosylate” refers to the group —O—S(O₂) -p-toluene.

The term “mesylate” refers to the group —O—S(O₂)-methyl.

The term “triflate” refers to the group —O—S(O₂)—CF₃.

The preferred embodiments provided above for R¹ and X of Formula I apply equally to R²¹ and X², respectively of Formula II.

In a preferred embodiment, the precursor compound of the invention is of Formula IIa:

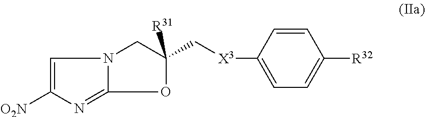

wherein:

R³¹ is as defined above for R²¹ of Formula II;

R³² is as defined above for R²² of Formula II;

X³ is as defined above for X² of Formula II.

Precursor compounds of the present invention may be obtained by following the methods described by Nagarajan et at (1989 Eur J Med Chem; 24: 631-633) by reaction of 2,4-dinitroimidazole (1) with a substituted oxirane (2) as illustrated in Scheme 1 below:

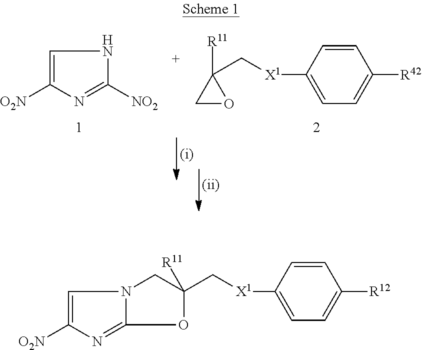

wherein _{R}¹¹, R¹² and X¹ are as suitably and preferably defined herein. R⁴² is either an R¹² group, or is an R¹² group protected by a suitable protecting group wherein the protecting group is removed in step (ii) of Scheme 1 following reaction in step (i) of 1 and 2 to obtain the precursor compound of the invention following deprotection. R⁴² may alternatively be a chemical group, or a suitably protected version thereof, which may be converted using known organic chemistry methods into an R¹² group in step (ii) following completion of step (i).

In an alternative, the precursor compounds of the invention may be obtained by following the methods described by Sasaki et at (2006 J Med Chem; 49 (26):7854-7860), wherein a 2-chloro-5-nitro imidazole starting material (3) is converted to the corresponding epoxide (4) and then reacted with the desired phenol (for X¹═—NH—) or phenylamine (for X¹═—NH—) (5) to obtain the precursor compound of the invention, as illustrated below in Scheme 2:

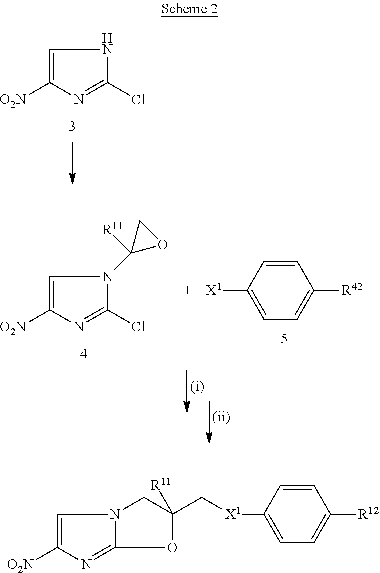

In Scheme 2, R¹¹, R¹², R⁴² and X¹ are as described above for Scheme 1.

The precursor compound of the invention is ideally provided in sterile, apyrogenic form. The precursor compound can accordingly be used for the preparation of a radiopharmaceutical composition comprising the compound of the invention wherein R² is a radioactive halogen, together with a biocompatible carrier suitable for mammalian administration, which forms another aspect of the invention as described in more detail below.

The precursor compound is also suitable for inclusion as a component in a kit or a cassette for the preparation of such a pharmaceutical composition. These aspects of the invention are also discussed in greater detail below.

Method to Prepare Compounds

With routine adaption, the above-described methods to obtain precursor compounds of the invention can also be applied to obtain a compound of Formula I wherein R² is a non-radioactive halogen isotope.

In another embodiment, the present invention relates to a method for the preparation of the compound of the invention wherein said compound comprises a radioactive halogen, and wherein said method comprises reaction of the precursor compound as defined herein with a suitable source of said radioactive halogen. The suitable and preferred aspects of the compound of Formula I and the precursor compound of Formula II as defined herein apply equally to this aspect of the invention.

The term “a suitable source said radioactive halogen” means the radioactive halogen in a chemical form that is reactive with a substituent of the precursor compound such that the radioisotope becomes covalently attached to the precursor compound. The person skilled in the art of in vivo imaging agents will be familiar with sources of radioactive halogen that are suitable for application in the present invention. The reader is referred to the “Handbook of Radiopharmaceuticals” for a detailed presentation of the field (2003; Wiley: Welch and Redvanly, Eds).

The step of “reaction” of the precursor compound with the suitable source of a radioactive halogen involves bringing the two reactants together under reaction conditions suitable for formation of the desired compound in as high a radiochemical yield (RCY) as possible. Synthetic routes for obtaining particular compounds of the present invention are presented in the experimental section below.

Methods of introducing radioactive halogens are described by Bolton (2002 J LabCompRadiopharm; 45: 485-528).

It is known in the art that to introduce a radioactive halogen (which can be either a gamma-emitting radioactive halogen or a positron-emitting radioactive halogen) the precursor suitably comprises the following reactive groups: a non-radioactive precursor halogen atom such as an aryl iodide or bromide (to permit radioiodine exchange); an activated aryl ring (e.g. phenol or aniline groups); an imidazole ring; an indole ring; an organometallic compound (eg. trialkyltin or trialkylsilyl); or an organic compound such as triazene or a good leaving group for nucleophilic substitution such as an iodonium salt. Methods of introducing radioactive halogens are described by Bolton (2002 J LabCompRadiopharm; 45: 485-528). Examples of suitable aryl groups to which radioactive halogens, especially iodine can be attached are given below:

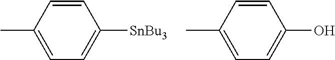

Both contain substituents which permit facile radioiodine substitution onto the aromatic ring. Alternative substituents containing radioactive iodine can be synthesised by direct iodination via radiohalogen exchange wherein radioiodide ion is the suitable source of radioactive iodine, e.g.:

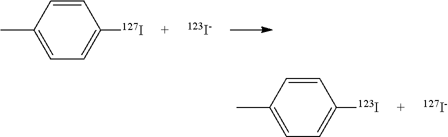

Where R² is radioactive iodine, a preferred precursor compound of Formula II comprises at R²² a derivative which either undergoes electrophilic iodination. Examples of this are organometallic derivatives such as a trialkylstannane (e.g. trimethylstannyl or tributylstannyl), or a trialkylsilane (e.g. trimethylsilyl) or an organoboron compound (e.g. boronate esters or organotrifluoroborates).

For electrophilic radioiodination, R²² of the precursor compound of Formula II preferably comprises: an activated organometallic precursor compound (e.g. trialkyltin, trialkylsilyl or organoboron compound). Precursor compounds and methods of introducing radioiodine into organic molecules are described by Bolton (2002 J Lab Comp Radiopharm; 45: 485-528). Suitable boronate ester organoboron compounds and their preparation are described by Kabalaka et at (2002 Nucl Med Biol; 29: 841-843 and 2003 Nucl Med Biol; 30: 369-373). Suitable organotrifluoroborates and their preparation are described by Kabalaka et at (2004 Nucl Med Biol 2004; 31: 935-938). Preferred precursor compounds of Formula II for radioiodination comprise at R²² an organometallic precursor compound, most preferably a trialkyltin, and especially tributyltin.

Radiobromination can be achieved by methods similar to those described above for radioiodination. Kabalka and Varma have reviewed various methods for the synthesis of radiohalogenated compounds, including radiobrominated compounds (1989 Tetrahedron; 45(21): 6601-21).

The methods used when the radioactive halogen is ¹⁸F are described in detail in Chapter 6 of the Handbook of Radiopharmaceuticals (2003; Wiley: Welch and Redvanly, Eds). ¹⁸F has a relatively short half-life and therefore special considerations are required in the synthesis of compounds comprising ¹⁸F.

Labelling with ¹⁸F can be achieved by nucleophilic displacement of a leaving group from a precursor compound. In this way, the precursor compound may be labelled in one step by reaction with a suitable source of [¹⁸F]-fluoride ion (¹⁸F), which is normally obtained as an aqueous solution from the nuclear reaction ¹⁸O(p,n)¹⁸F and which is made reactive by the addition of a cationic counterion and the subsequent removal of water to form a suitable source of ¹⁸F. ^{T}he radiofluorine atom attaches via a direct covalent bond to the aromatic ring. ¹⁸F-fluoride nucleophilic displacement from an aryl diazonium salt, aryl nitro compound or an aryl quaternary ammonium salt are suitable routes. Preferably, where it is desired to add radioactive fluorine, R²² of said precursor compound is a leaving group selected from hydroxyl, nitro, bromo, iodo, tri-C₁₋₃-alkylammonium, quaternary ammonium, diazonium, iodonium, tosylate, mesylate and triflate, and said suitable source of radioactive halogen is ¹⁸F-fluoride (¹⁸F⁻).

In one embodiment, the method for the preparation is automated. A cassette useful in this automated method forms a further aspect of the invention describe in more detail below.

Kit and Cassette

In a yet further aspect, the present invention provides a kit for the preparation of a compound of the invention wherein R² is a radioactive halogen, said kit comprising a precursor compound of the invention as defined herein, so that reaction with a sterile source of a radioactive halogen gives the desired compound with the minimum number of manipulations. Such considerations are particularly important where the radioisotope has a relatively short half-life, and for ease of handling and hence reduced radiation dose for the radiopharmacist. The precursor compound is preferably present in the kit in lyophilized form, and the reaction medium for reconstitution of such kits is preferably a biocompatible carrier. Suitable and preferred embodiments of the precursor compound for the kit of the invention are as provided above for the precursor compound of the invention.

A “biocompatible carrier” is a fluid, especially a liquid, in which the resultant radiolabelled compound of the invention is suspended or dissolved, such that the composition is physiologically tolerable, i.e. can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). The biocompatible carrier may also comprise biocompatible organic solvents such as ethanol. Such organic solvents are useful to solubilise more lipophilic compounds or formulations. Preferably the biocompatible carrier comprises pyrogen-free water for injection, or isotonic saline. The pH of the biocompatible carrier for intravenous injection is suitably in the range 4.0 to 10.5.

In the kit of the invention, the precursor compound is preferably presented in a sealed container which permits maintenance of sterile integrity and/or radioactive safety, plus optionally an inert headspace gas (e.g. nitrogen or argon), whilst permitting addition and withdrawal of solutions by syringe. A preferred sealed container is a septum-sealed vial, wherein the gas-tight closure is crimped on with an overseal (typically of aluminium). Such sealed containers have the additional advantage that the closure can withstand vacuum if desired e.g. to change the headspace gas or degas solutions.

The precursor compound for use in the kit may be employed under aseptic manufacture conditions to give the desired sterile, non-pyrogenic material. The precursor compound may alternatively be employed under non-sterile conditions, followed by terminal sterilisation using e.g. gamma-irradiation, autoclaving, dry heat or chemical treatment (e.g. with ethylene oxide). Preferably, the precursor compound is provided in sterile, non-pyrogenic form. Most preferably the sterile, non-pyrogenic precursor compound is provided in the sealed container as described above.

Preferably, all components of the kit are disposable to minimise the possibilities of contamination between runs and to ensure sterility and quality assurance.

[¹⁸F]-radiotracers in particular are now often conveniently prepared on an automated radiosynthesis apparatus. There are several commercially-available examples of such apparatus, including Tracerlab™ and Fastlab™ (GE Healthcare Ltd). Such apparatus commonly comprises a “cassette”, often disposable, in which the radiochemistry is performed, which is fitted to the apparatus in order to perform a radiosynthesis. The cassette normally includes fluid pathways, a reaction vessel, and ports for receiving reagent vials as well as any solid-phase extraction cartridges used in post-radiosynthetic clean up steps.

The present invention therefore provides in another aspect a cassette for the automated synthesis of compound of Formula I comprising ¹⁸F, wherein said cassette comprises:
(i) a vessel containing a precursor compound comprising a leaving group wherein said leaving group is as defined herein for the precursor compound of the invention; and
(ii) means for eluting the vessel with a suitable source of ¹⁸F-fluoride (¹⁸F⁻).

The cassette may additionally comprise:
(iii) an ion-exchange cartridge for removal of excess ¹⁸F-fluoride (¹⁸F⁻).

Pharmaceutical Composition

In another aspect, the present invention provides a pharmaceutical composition comprising the compound of Formula I together with a biocompatible carrier in a form suitable for mammalian administration.

When R² of the compound of Formula I in said pharmaceutical composition is a radioactive halogen, said pharmaceutical composition is a radiopharmaceutical composition and the biocompatible carrier is as defined above in relation to the kit of the invention. The radiopharmaceutical composition may be administered parenterally, i.e. by injection, and is most preferably an aqueous solution. Such a composition may optionally contain further ingredients such as buffers; pharmaceutically acceptable solubilisers (e.g. cyclodextrins or surfactants such as Pluronic, Tween or phospholipids); pharmaceutically acceptable stabilisers or antioxidants (such as ascorbic acid, gentisic acid orpara-aminobenzoic acid). Where the compound of the invention is provided as a radiopharmaceutical composition, the method for preparation of said compound may further comprise the steps required to obtain a radiopharmaceutical composition, e.g. removal of organic solvent, addition of a biocompatible buffer and any optional further ingredients. For parenteral administration, steps to ensure that the radiopharmaceutical composition is sterile and apyrogenic also need to be taken.

The suitable and preferred embodiments described herein for the compound of Formula I wherein R² is a radioactive halogen apply equally to the radiopharmaceutical composition of the invention.

Where the pharmaceutical composition comprises the compound of Formula I wherein R² is a non-radioactive halogen, the biocompatible carrier may be a solid or liquid pharmaceutically acceptable nontoxic carrier. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerols solutions are also be employed as liquid carriers, particularly for injectable solutions. Suitable pharmaceutical excipients include starch, glucose, lactose, sucrose, gelatine, malt, rice, flour, chalk, silica gel, magnesium carbonate, magnesium stearate, sodium stearate, glycerol monostearate, talc, sodium chloride, dried skim milk, glycerol, propylene glycol, water, ethanol and the like. These compositions can take the form of solutions, suspensions, tablets, pills, capsules, powders, sustained release formulations and the like. Suitable pharmaceutical carriers are described in “Remington's Pharmaceutical Sciences” (18^{th} Edition; E. W. Martin, Ed: 1990 Mack Publishing). Such compositions will contain an effective therapeutic amount of the compound together with a suitable amount of carrier so as to provide the form for proper administration to the host. While intravenous injection is a very effective form of administration, other modes can be employed, e.g. oral administration.

In Vivo Imaging and Diagnosis

In a further aspect, the present invention provides an in vivo imaging method comprising:
(a) administration of the compound of Formula I wherein R² is a radioactive halogen;
(b) allowing said compound to bind to the cell wall of any mycobacteria present in said subject;
(c) detecting by an appropriate in vivo imaging procedure signals emitted by the radioactive halogen comprised in said compound;
(d) generating an image representative of the location and/or amount of said signals; and,
(e) determining the distribution of mycobacteria in said subject wherein said distribution is directly correlated with said signals emitted by said radioactive halogen.

The “administration” step is preferably carried out parenterally, and most preferably intravenously. The intravenous route represents the most efficient way to deliver the compound throughout the body of the subject, and also does not represent a substantial physical intervention on the body of the subject. By the term “substantial” is meant an intervention which requires professional medical expertise to be carried out, or which entails a substantial health risk even when carried out with the required professional care and expertise. The compound is preferably administered as the pharmaceutical composition of the invention, as defined herein. The in vivo imaging method of the invention can also be understood as comprising the above-defined steps (b)-(e) carried out on a subject to whom said compound has been pre-administered. In this embodiment, the compound is preferably administered as the radiopharmaceutical composition of the invention.

Following the administering step and preceding the detecting step, the compound is allowed to bind to mycobacteria within said subject. For example, when the subject is an intact mammal, the compound will dynamically move through the mammal's body, coming into contact with various tissues therein. Once the compound comes into contact with any mycobacteria, the two entities bind such that clearance of the compound from tissue in which mycobacteria are present takes longer than from tissue without any mycobacteria present. A certain point in time will be reached when detection of compound specifically bound to mycobacteria is enabled as a result of the ratio between compound bound to tissue with mycobacteria versus that bound in tissue without any mycobacteria. This is the optimal time for the detecting step to be carried out.

The “detecting” step of the method of the invention involves detection of signals emitted by the radioactive halogen by means of a detector sensitive to said signals. This detection step can also be understood as the acquisition of signal data. Single-photon emission tomography (SPECT) and positron-emission tomography (PET) are suitable in vivo imaging procedures for use in the method of the invention. When R² is a gamma-emitting radioactive halogen, SPECT is suitable, and when R² is a positron-emitting radioactive halogen, PET is suitable.

The “generating” step of the method of the invention is carried out by a computer which applies a reconstruction algorithm to the acquired signal data to yield a dataset. This dataset is then manipulated to generate images showing the location and/or amount of signals emitted by the radioactive halogen which is comprised in the compound used in said in vivo imaging method. The signals emitted directly correlate with the presence of mycobacteria such that the “determining” step can be made by evaluating the generated image.

The “subject” of the invention can be any human or animal subject. Preferably the subject of the invention is a mammal. Most preferably, said subject is an intact mammalian body in vivo. In an especially preferred embodiment, the subject of the invention is a human. The in vivo imaging method may be used in subjects known or suspected to have a pathological condition associated with a mycobacterial infection. Preferably, said method relates to the in vivo imaging of a subject known or suspected to have tuberculosis caused by Mycobacrerium tuberculosis, and therefore has utility in a method for the diagnosis of said condition. Where a subject is known to have tuberculosis caused by Mycobacrerium tuberculosis, the in vivo imaging method of the invention may be carried out repeatedly during the course of a treatment regimen for said subject, said regimen comprising administration of a drug to combat tuberculosis caused by Mycobacrerium tuberculosis.

The present invention additionally provides a method for diagnosis of a mycobacterial infection in a subject wherein said method comprises the in vivo imaging method as defined herein, together with a further step (vi) of attributing the distribution of mycobacteria to a mycobacterial infection. The term “mycobacterial infection” is defined herein as an infection caused by a mycobacterium. The method of diagnosis is preferably used to diagnose tuberculosis caused by Mycobacterium tuberculosis.

In a yet further aspect, the present invention provides the radiopharmaceutical composition as suitably and preferably defined herein for use in a method of in vivo imaging wherein said method of in vivo imaging is as suitably and preferably defined herein.

The present invention also provides the radiopharmaceutical composition as suitably and preferably defined herein for use in a method of diagnosis wherein said method of diagnosis is as suitably and preferably defined herein.

Treatment

In a yet further aspect, the present invention provides a method for the treatment of a mycobacterial infection comprising administration of the compound of Formula I wherein R² is a non-radioactive halogen. Preferably, said compound is administered as a pharmaceutical composition. A suitable pharmaceutical composition for a compound of Formula I wherein R² is a non-radioactive halogen is defined above. As for the methods of in vivo imaging and diagnosis of the invention, said mycobacterial infection is preferably tuberculosis caused by

Mycobacterium tuberculosis.

As presented in the experimental examples herein, the compound of Formula I of the present invention wherein R² is a non-radioactive halogen has good activity against Mycobacterium tuberculosis and as such has properties which make it a potentially useful treatment against Mycobacterium tuberculosis.

The suitable and preferred embodiments of R² as a non-radioactive halogen as presented above in connection with the compound of Formula I apply equally to the method of treatment of the invention.

In one embodiment, the method of treatment may also comprise the combined administration of the compound of the invention with other known treatments for tuberculosis. Non-limiting examples of such other treatments including isoniazid, rifampicin, pyrazinamide, and ethambutol.

### BRIEF DESCRIPTION OF THE EXAMPLES

Example 1 describes the synthesis of the unlabelled prior art compound, (R)-2-Methyl-6-nitro-2-(phenoxymethyl)-2,3-dihydroimidazo[2,1-b]oxazole.

Example 2 describes the synthesis of an iodinated version of the prior art compound prepared in Example 1, (R)-2-((4-Iodophenoxy)methyl)-2-methyl-6-nitro-2,3-dihydroimidazo[2,1-b]oxazole, a compound of Formula I of the invention wherein R² is non-radioactive iodine.

Example 3 describes the in vitro screening methods used to evaluate the compounds obtained in Examples 1 and 2.

Example 4 describes the synthesis of (R)-2-((4-fluorophenoxy)methyl)-2-methyl-6-nitro-2,3-dihydroimidazo[2,1-b]oxazole, a compound of Formula I of the invention wherein R² is non-radioactive fluorine.

Example 5 describes the synthesis of (R)-2-methyl-6-nitro-2-((4-(tributylstannyl)phenoxy)methyl)-2,3-dihydroimidazo[2,1-b]oxazole, a precursor compound of the invention.

### LIST OF ABBREVIATIONS USED IN THE EXAMPLES

ATP adenosine triphosphate
DCM dichloromethane
DMF dimethylformamide
HPLC high-performance liquid chromatography
IC50 half maximal inhibitory concentration
LCMS liquid chromatography mass spectrometry
LORA low-oxygen recovery assay
MABAmicroplate alamar blue assay
MIC minimum inhibitory concentration
RBF round-bottom flask
VERO “verda reno” meaning “green kidney” in Esperanto; used to refer to a line of kidney epithelial cells extracted from an African green monkey (Cercopithecus aethiops).

### EXAMPLES

### Example 1

### Synthesis of (R)-2-Methyl-6-nitro-2-(phenoxymethyl)-2,3-dihydroimidazo[2,1-bloxazole (Prior Art Compound)

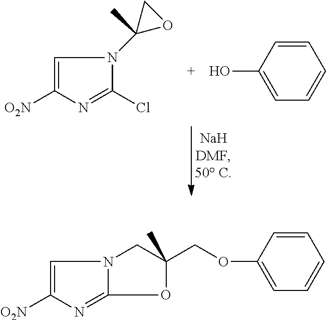 [SMILES: C[C@@]1(n2cc([N+](=O)[O-])nc2Cl)CO1 ; Oc1ccccc1 ; C[C@]1(COc2ccccc2)Cn2cc([N+](=O)[O-])nc2O1]

(R)-2-chloro-1- (2-methyl-2,3-epoxypropyl)-4-nitroimidazole was obtained by conversion of commercially-available 2-chloro-5-nitro imidazole starting material to the corresponding epoxide following the method described by Sasaki et at (2006 J Med Chem; 49: 7854-7860). (R)-2-chloro-1- (2-methyl-2,3-epoxypropyl)-4-nitroimidazole (57.7 mg, 0.267 mmol), and phenol (20.12 mg, 0.214 mmol) were placed in a 50 ml RBF and dissolved in 2 ml of DMF. The reaction mixture was cooled to 0° C. and then to it, NaH (16.48 mg, 0.256 mmol) was added carefully. The temperature was then increased to 50° C. and the reaction mass was stirred for 24-36 hours. The reaction was checked for completion using the HPLC/LCMS and the reaction mass was concentrated on a rotary evaporator. The dried material was then taken for purification on a CombiFlash (Teledyne Isco) chromatography system (using a DCM-methanol solvent system). The purified material was then taken for recrystallization using a DCM-hexane solvent system to yield a pale yellow powder as the product. Yield=7.4 mg; Purity=96%; ¹H NMR (CDCl3): δ 1.8(dd (J=3.0,9.0), 2H,CH₂), 4.22 (d (J=9), 1H, CH2), 4.5 (d (J=9), 1H, CH2), 6.86 (d (J=9.0), 2H, ArH), 7.6 (t (J=6.0, 1H, ArH), 7.33 (d (J=6.0), 2H, ArH), 7.6 (s, 1H, ArH); MS: m/z 276 (M+1, 100%).

### Example 2

### Synthesis of (R)-2-((4-Iodophenoxy)methyl)-2-methyl-6-nitro-2,3-dihydroimidazol[2,1-b]oxazole (iodinated derivative of the prior art compound of Example 1)

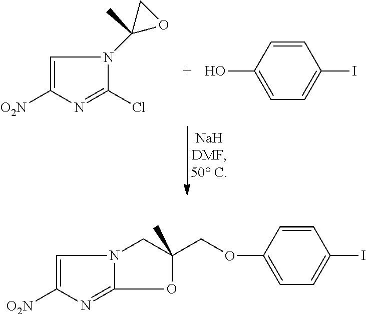 [SMILES: C[C@@]1(n2cc([N+](=O)[O-])nc2Cl)CO1 ; Oc1ccc(I)cc1 ; C[C@]1(COc2ccc(I)cc2)Cn2cc([N+](=O)[O-])nc2O1]

The method as described in Example 1 was used except that p-iodo phenol (28.38 mg, 0.129 mmol) was used in place of phenol. Yield=4.2 mg; Purity=96%; 1H NMR (CDCl3): δ 1.8(dd (J=3.0,9.0), 2H,CH2), 4.22 (d (J=9), 1H, CH2), 4.5 (d (J=9), 1H, CH2), 6.64 (d (j=9.0), 2H, ArH), 7.6 (m, 3H, ArH) ; MS: m/z 402 (M+1, 100%).

### Example 3

### Methods used to Screen Compounds In Vitro

3(i)Methods for Determining Minimum Inhibition Concentration (MIC)

Screening was done to get MIC for M. tuberculosis using both the microplate alamar blue assay (MABA) and low-oxygen recovery assay (LORA).

The initial screen was conducted against Mycobacterium tuberculosis strain H37Rv (American Type Culture Collection number 27294) in BACTEC 12B medium (Becton-Dickinson) using the MABA. Compounds were tested in ten 2-fold dilutions, typically from 100 μg/mL to 0.19 μg/mL. The MIC90 is defined as the concentration effecting a reduction in fluorescence of 90% relative to controls. This value is determined from the dose-response curve using a curve-fitting program. Any MIC90 value of ≦10 μg/mL was considered “active” for antitubercular activity. 3(h) Method for Determining IC50

A VERO cell cytotoxicity assay was carried out in parallel with the TB Dose Response assay. After 72 hours exposure, viability was assessed using Promega's Cell Titer Glo Luminescent Cell Viability Assay, a homogeneous method of determining the number of viable cells in culture based on quantitation of the ATP present. Cytotoxicity was determined from the dose-response curve as the IC50 using a curve-fitting program.

3(iii) Method for Determining Calculated clogP

Chemdraw Ultra 10.0 (Cambridge Soft Software) was used to determine calculated clogP values.

3(iv) In Vitro Screening Results

| Compound | MABA MIC (μg/ml) | IC50(μg/ml) | Calcd clogP |
|---|---|---|---|
| Example 1 | 1.329 | >50 | 2.4 |
| Example 2 | <0.195 | >50 | 3.63 |

The above screening data demonstrates that introduction of iodine has reduced the MIC, by a factor of over 6 which means iodine introduction has surprisingly increased the activity of the parent compound.

### Example 4

### Synthesis of (R)-2-((4-fluorophenoxy)methyl)-2-methyl-6-nitro-2,3-dihydroimidazo[2,1-b]oxazole

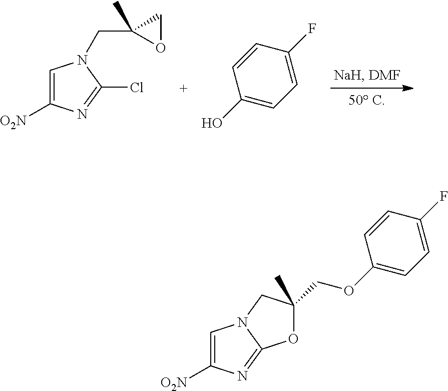 [SMILES: C[C@@]1(Cn2cc([N+](=O)[O-])nc2Cl)CO1 ; Oc1ccc(F)cc1 ; C[C@]1(COc2ccc(F)cc2)Cn2cc([N+](=O)[O-])nc2O1]

(R)-2-chloro-1-((2-methyloxiran-2-yl)methyl)-4-nitro-1H-imidazole (50.0 mg, 0.230 mmol) was transferred to clean, dry RBF and to it, added anhydrous DMF (2.0 ml). To this mass, added p-fluoro phenol (20.66 mg, 0.184 mmol) and stirred under nitrogen for 10 minutes. The mixture was then cooled to 0° C. and then added sodium hydride (60%) (8.84 mg, 0.221 mmol) portion wise. The contents of the flask were allowed to stir in cold conditions for about 10 minutes and then heated to 50° C. The reaction showed completion within 30 hours on the LC/MS. The contents of the flask were allowed to cool to room temperature and then concentrated on the rotary evaporator. The resulting mass as such was taken for purification on the CombiFlash system using DCM/Methanol as the gradient system. The resulting solid was then recrystallized using a DCM/Hexane system to yield 5 mg (74.6%) of the product as a whitish solid.

LC-MS: m/z calcd for C13H12FN3O4, 293.08; found, 294 (M+H).

### Example 5

### Synthesis of (R)-2-methyl-6-nitro-2-((4-(tributylstannyl)phenoxy)methyl)-2,3-dihydromidazo[2,1-b]oxazole

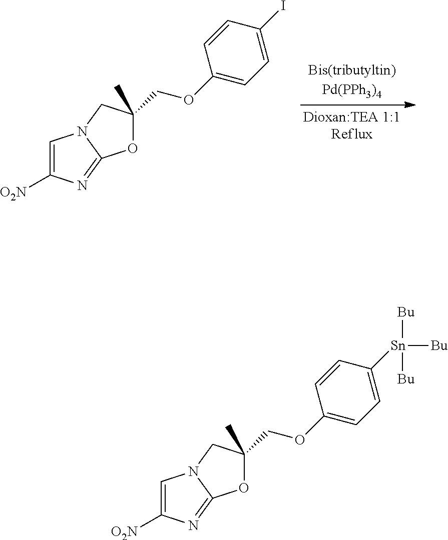

A mixture of (R)-2-((4-iodophenoxy)methyl)-2-methyl-6-nitro-2,3-dihydroimidazo[2,1-b]oxazole prepared according to Example 2 (25 mg, 0.0623 mmol), bis- (tributyltin) (54.25 mg, 47 μl, 0.0935 mmol) and tetrakis triphenylphosphine) palladium (0) (5.11 mg, 0.004426 mmol) was taken in a mixed solvent (2.0 ml, 1:1 dioxane/triethyl amine) and stirred under reflux for 36 hours. Upon checking for completion, the solvent was removed, and to the residue added 4-5 ml of water. The reaction mixture was then extracted using ethyl acetate, separated, dried and evaporated. The residue was then purified using the HPLC system. However, once purified, the compound could not be isolated from the solvent as the molecule is not stable once removed from it. The confirmation of product formation was from the LC/MS system with a single peak with m/z 565 (M+H)⁻.

1. A compound of Formula I: 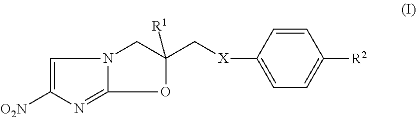
wherein:
R¹ is absent or is C₁₋₄ alkyl;
R² is a radioactive halogen; and,
X is —O— or —NH—.

2. The compound as defined in claim 1 wherein R¹ is methyl.

3. The compound as defined in claim 1 wherein X is —O—.

4. (canceled)

5. The compound as defined in claim 1 wherein said radioactive halogen is a gamma-emitting radioactive halogen selected from ¹²³I, ¹³¹I and ⁷⁷Br.

6. (canceled)

7. The compound as defined in claim 1 wherein said radioactive halogen is a positron-emitting radioactive halogen selected from ¹⁷F, ¹⁸F, ⁷⁵Br, ⁷⁶Br and ¹²⁴I.

8.-10. (canceled)

11. The compound as defined in claim 1 which is of Formula Ia: 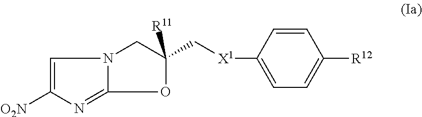
wherein
R¹¹ is as defined for R¹ in claim 1;
R¹² is as defined for R² claim 1; and ,
X¹ is as defined for X group in claim 1.

12. A precursor compound for the preparation of compound of Formula I as defined in claim 1, which is a compound of Formula II: 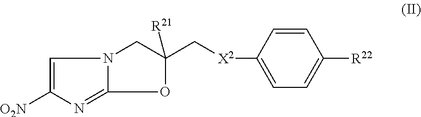
wherein:
R²¹ is as defined for R¹ in claim 1;
R²² is a non-radioactive iodine or bromine, an organometallic derivative such as a trialkylstannane or a trialkylsilane, an organoboron compound such as a boronate ester or an organotrifluoroborate, or is selected from amino, hydroxy, nitro, bromo, iodo, tri-C₁₋₃-alkylammonium, quaternary ammonium, diazonium, iodonium, tosylate, mesylate and triflate; and,
X² is as defined for X in claim 1.

13. The precursor compound as defined in claim 12 which is of Formula IIa: 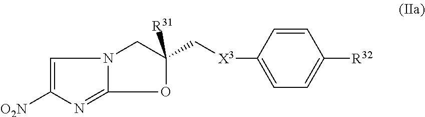
wherein:
R³¹ is as defined for R²¹ in claim 12;
R³² is as defined for R²² in claim 12;
X³ is as defined for X² in claim 12.

14. A method for the preparation of a compound of Formula I: 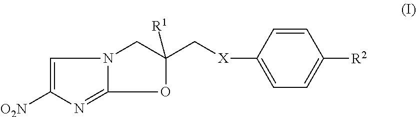
wherein:
R¹ is absent or is C₁₋₄ alkyl;
R² is a radioactive halogen; and,
X is —O— or —NH—;
wherein said method comprises reaction of the precursor compound as defined in claim 12 with a suitable source of said radioactive halogen.

15.-19. (canceled)

20. A pharmaceutical composition comprising the compound as defined in claim 1 together with a biocompatible carrier in a form suitable for mammalian administration.

21. An in vivo imaging method comprising:
(a) administration of the compound as defined in claim 1;
(b) allowing said compound to bind to the cell wall of any mycobacteria present in said subject;
(c) detecting by an in vivo imaging procedure signals emitted by said radioactive halogen;
(d) generating an image representative of the location and/or amount of said signals; and,
(e) determining the distribution of mycobacteria in said subject wherein said distribution is directly correlated with said signals.

22. (canceled)

23. The in vivo imaging method as defined in claim 21 wherein said mycobacterium is Mycobacterium tuberculosis.

24. The in vivo imaging method as defined in claim 23 which is carried out repeatedly during the course of a treatment regimen for said subject, said regimen comprising administration of a drug to combat tuberculosis caused by Mycobacterium tuberculosis.

25. A method for diagnosis of a mycobacterial infection in a subject wherein said method comprises the in vivo imaging method as defined in claim 21, together with a further step (vi) of attributing the distribution of mycobacterium to a mycobacterial infection.

26. The method of diagnosis as defined in claim 25 wherein said mycobacterial infection is tuberculosis caused by Mycobacterium tuberculosis.

27.-29. (canceled)
